(19) United States
(12) Patent Application Publication
(10) Pub. No.: US 2006/0014782 A1
(43) Pub. Date: Jan. 19, 2006

(54) Use of pde iv inhibitors to treat angiogenesis

(76) Inventors: Daniel A. Gamache, Arlington, TX (US); David P. Bingaman, Weatherford, TX (US); Michael A. Kapin, Arlington, TX (US)

Correspondence Address: Alcon Research, 6201 South Freeway, Fort Worth, TX, 76134-2099, US

(21) Appl. No.: 10/527,599

(22) PCT Filed: Sep. 11, 2003
(86) PCT No.: PCT/US03/28675

§ 371 Date: Mar. 14, 2005

Related U.S. Application Data
(60) Provisional application No. 60/411,001, filed on Sep. 16, 2002.

(51) Int. Cl. A61K 31/519

(52) U.S. Cl. 514/310; 514/252.16; 514/262.1

(57) ABSTRACT

Selective PDE-IV inhibitors are useful for preventing and treating angiogenic/edema related diseases and disorders.

The present invention is directed to the prevention and treatment of angiogenic and edematous disorders of the eye. In particular, the present invention is directed to the use of phosphodiesterase type-IV (PDE-IV) inhibitors in the treatment of ocular angiogenic and edematous disorders in mammals.

### BACKGROUND OF THE INVENTION

There are many agents known to inhibit the formation of new blood vessels (angiogenesis). For example, steroids functioning to inhibit angiogenesis in the presence of heparin or specific heparin fragments are disclosed in Crum, et al., A New Class of Steroids Inhibits Angiogenesis in the Presence of Heparin or a Hepatin Fragment, Science, Vol. 230:1375-1378, Dec. 20, 1985. The authors is refer to such steroids as “angiostatic” steroids. Included within this class of steroids found to be angiostatic are the dihydro and tetrahydro metabolites of cortisol and cortexolone. In a follow-up study directed to testing a hypothesis as to the mechanism by which the steroids inhibit angiogenesis, it was shown that heparin/angiostatic steroid compositions cause dissolution of the basement membrane scaffolding to which anchorage dependent endothelia are attached resulting in capillary involution; see, Ingber, et al., A Possible Mechanism for Inhibition of Angiogenesis by Angiostatic Steroids: Induction of Capillary Basement Membrane Dissolution, Endocrinology Vol. 119:1768-1775, 1986.

A group of tetrahydro steroids useful in inhibiting angiogenesis is disclosed in U.S. Pat. No. 4,975,537, Aristoff, et al. The compounds are disclosed for use in treating head trauma, spinal trauma, septic or traumatic shock, stroke, and hemorrhage shock. In addition, the patent discusses the utility of these compounds in embryo implantation and in the treatment of cancer, arthritis, and arteriosclerosis. Some of the steroids disclosed in Aristoff et al. are disclosed in U.S. Pat. No. 4,771,042 in combination with heparin or a heparin fragment for inhibiting angiogenesis in a warm blooded animal.

Compositions of hydrocortisone, “tetrahydrocortisol-S,” and U-72,745G, each in combination with a beta cyclodextrin, have been shown to inhibit corneal neovascularization: Li, et al., Angiostatic Steroids Potentiated by Sulphated Cyclodextrin Inhibit Corneal Neovascularization, Investigative Ophthalmology and Visual Science, Vol. 32(11):2898-2905, October, 1991. The steroids alone reduce neovascularization somewhat but are not effective alone in effecting regression of neovascularization.

Tetrahydrocortisol (THF) has been disclosed as an angiostatic steroid in Folkman, et al., Angiostatic Steroids, Ann. Surg., Vol. 206(3), 1987, wherein it is suggested angiostatic steroids may have potential use for diseases dominated by abnormal neovascularization, including diabetic retinopathy, neovascular glaucoma, and retrolental fibroplasia.

It has been previously shown that certain nonsteroidal antiinflammatory drugs (NSAIDs) can inhibit angiogenesis and vascular edema in pathologic conditions. The ability of most NSAIDs to influence vascular permeability and angiogenesis appears to be associated with their ability to block the cyclo-oxygenase enzymes (COX-1 and -2). Blockade of COX-1 and -2 is associated with a decrease in inflammatory mediators, such as PGE₂. Moreover, it appears that PGE₂ inhibition results in decreased expression and production of various cytokines including vascular endothelial growth factor (VEGF). VEGF is known to produce vascular leakage and angiogenesis in the eye of preclinical models. Also, increased levels of VEGF have been found in neovascular tissues and extracellular fluid from the eyes of patients with diabetic retinopathy and age-related macular degeneration. Thus, NSAIDs may inhibit vascular leakage and angiogenesis by modulating PGE₂ levels and its effects on VEGF expression and activity. This theory is supported by work involving animal tumor models which demonstrate that systemic administration of COX-2 inhibitors decreases PGE₂ and VEGF tissue levels and thereby prevent tumor-induced angiogenesis. In these models, VEGF activity and angiogenesis are restored by adding exogenous PGE₂ during continued COX-2 blockade. However, NSAIDs appear to have variable activity in animal models of ocular neovascularization (NV), where selective COX inhibitors have shown disparate activity against preretinal NV and/or CNV.

As described in commonly owned U.S. application Ser. No. 09/929,381, it was found that certain 3-benzoylphenlacetic acids and derivatives, which are NSAIDs, are useful for treating angiogenesis-related disorders.

PDE-IV belongs to a family of cyclic nucleotide hydrolyzing enzymes which are distinguished by substrate preference, tissue distribution, and biochemical and pharmacological properties. PDE-I enzymes are Calcium/calmodulin dependent, PDE-II enzymes are cGMP-stimulated, PDE-III enzymes are cGMP inhibited, PDE-IV enzymes are cAMP specific, PDE-V are cGMP specific, PDE-VI exists only in the retina, and PDE-VII enzymes have a high affinity for cAMP. Selective inhibitors of individual phosphodiesterase enzymes can be identified in in vitro enzyme assays using known techniques. Since PDE-IV activity controls the levels of cAMP in inflammatory cells, inhibitors of this enzyme have anti-inflammatory activity. Inhibitors of phosphodiesterases vary in selectivity and specificity for individual enzymes and therefore can possess diverse pharmacological and toxicological properties.

It has been reported that leukocyte adhesion is a key early event in early corneal angiogensis (Becker, et al., IOVS, 1999, Vol. 40(3):612-618) and in vascular disorders of the retina such as seen in models of diabetic retinopathy (Adamis, A. P., et al., IOVS, 2000, Vol. 41(4):S406). The process of leukocyte adheshion is primarly mediated by leukocyte integrins and intercellular adhesion molecule-1 on the endothelial surface. PDE-IV inhibitors prevent leukocyte adhesion by suppressing endothelial cell ICAM-1 expression by inhibiting leukocyte activation, see, for example, J. Neuroimmanol., 1998, Vol. 89(1-2):97-103. Also, PDE-IV inhibitors have been reported to suppress release of cytokines and eicosanoids from endothelial and epithelial cells. Therefore, PDE-IV inhibitors decrease the release of a variety of pro-inflammatory and pro-angiogenic mediators derived from several cell types.

### SUMMARY OF THE INVENTION

The present invention is directed to the prevention and treatment of diseases and disorders of the eye involving angiogenesis and edema, using PDE-IV inhibitors.

### DETAILED DESCRIPTION OF THE INVENTION

Posterior segment neovascularization is the vision-threatening pathology responsible for the two most common causes of acquired blindness in developed countries: exudative age-related macular degeneration (AMD) and proliferative diabetic retinopathy (PDR). Currently the only approved treatments for posterior segment NV that occurs in exudative AMD is laser photocoagulation or photodynamic therapy with Visudyne®; both therapies involve occlusion of affected vasculature which results in localized laser-induced damage to the retina. Surgical interventions with vitrectomy and membrane removal are the only options currently available for patients with proliferative diabetic retinopathy. No strictly pharmacologic treatment has been approved for use against posterior segment NV, although several different compounds are being evaluated clinically, including, for example, anecortave acetate (Alcon Research, Ltd.), Macugen (Eyetech/Pfizer), Lucentis (Genentech/Novartis), squalamine (Genaera), and adPEDF (GenVec) for AMD and LY333531 (Lilly) and Fluocinolone (Bausch & Lomb) for diabetic macular edema.

In addition to changes in the retinal microvasculature induced by hyperglycemia in diabetic patients leading to macular edema, proliferation of neovascular membranes is also associated with vascular leakage and edema of the retina. Where edema involves the macula, visual acuity worsens. In diabetic retinopathy, macular edema is the major cause of vision loss. Like angiogenic disorders laser photocoagulation is used to stabilize or resolve the edematous condition. Unfortunately, laser photocoagulation is a cytodestructive procedure, that while preventing further edema to develop, will alter the visual field of the affected eye.

An effective pharmacologic therapy for posterior segment NV and edema would likely provide substantial efficacy to the patient, thereby avoiding invasive surgical or damaging laser procedures. Effective treatment of the NV would improve the patient's quality of life and productivity within society. Also, societal costs associated with providing assistance and health care to the blind could be dramatically reduced.

This invention applies to inhibitors of the PDE type-IV enzyme with the primary biological effect being suppression of NV. Selective inhibitors of the PDE type-IV enzyme are preferred. As used herein, “selective PDE-IV inhibitor” means a non-steroid compound that selectively inhibits type IV phosphodiesterase enzyme activity (relative to activities of other types of phosphodiesterase enzymes). As used herein, a compound that selectively inhibits type IV phosphodiesterase enzyme activity is a compound that is at least ten times more potent at inhibiting type IV phosphodiesterase enzyme activity than any other type of phosphodiesterase enzyme activity. Preferred PDE-IV inhibitors for use in the present invention are at least one thousand times more potent at inhibiting type IV phosphodiesterase enzyme activity than any other type of phosphodiesterase enzyme activity.

Selective PDE-IV inhibitors are known. Examples of selective PDE-IV inhibitors useful in the methods of the present invention include, but are not limited to: 2-(4-ethoxycarbonylaminobenzyl)-6-(3,4-dimethoxyphenyl)-2,3,4,5-tetrahydro-pyridazin-3-one and the related compounds disclosed in EP 0 738 15; 3-[3-(cyclopentyloxy)-4-methoxybenzyl]-6-(ethylamino)-8-isopropyl-3H-purine hydrochloride (also known as V-11294A) and the related compounds disclosed in WO 96/00218; 8-methoxyquinoline-5-[N-(2,5-dichloropyridin-3-yl)]carboxamide (also known as D-4418) and related compounds disclosed in WO 96/36595; the compounds disclosed in U.S. Pat. No. 5,605,914; cipamfylliine (also known as BRL-61063); ariflo (also known as SB-207499); and compounds disclosed in WO 99/50270.

According to the methods of the present invention, a composition comprising one or more selective PDE-IV inhibitors and a pharmaceutically acceptable carrier for systemic or local administration is administered to a mammal in need thereof. The compositions are formulated in accordance with methods known in the art for the particular route of administration desired.

The PDE-IV inhibitors of the present invention can be administered either systemically or locally. Systemic administration includes: oral, transdermal, subdermal, intraperitioneal, subcutaneous, transnasal, sublingual, or rectal. Preferred administration is oral. Local administration for ocular administration includes: topical, intravitreal, periocular, transcleral, retrobulbar, sub-tenon, or via an intraocular device.

The compositions administered according to the present invention comprise a pharmaceutically effective amount of one or more selective PDE-IV inhibitors. As used herein, a “pharmaceutically effective amount” is one which is sufficient to reduce or prevent NV and/or edema. Generally, for compositions intended to be administered systemically for the treatment of ocular NV the total amount of selective PDE-IV inhibitor will be about 0.01-100 mg/kg.

The preferred compositions of the present invention are intended for administration to a human patient suffering from a NV disease or edematous disorder, such as, diabetic retinopathy, chronic glaucoma, retinal detachment, sickle cell retinopathy, age-related macular degeneration, rubeosis iritis, uveitis, neoplasms, Fuch's heterochromic iridocyclitis, neovascular glaucoma, corneal neovascularization, neovascularization resulting from combined vitrectomy and lensectomy, retinal ischemia, choroidal vascular insufficiency, choroidal thrombosis, carotid artery ischemia, contusive ocular injury, and retinopathy of prematurity.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

What is claimed is:

1. A method for treating angiogenic/edema-related diseases and disorders of the retina which comprises, administering a pharmaceutically effective amount of a selective PDE-IV inhibitor.
